(19) United States
(12) Patent Application Publication
(10) Pub. No.: US 2021/0155950 A1
(43) Pub. Date: May 27, 2021

(54) GENE OSNIA3 OF RICE NITRATE REDUCTASE NIA3 PROTEIN, AND ITS APPLICATION

(71) Applicant: Jiangxi Agricultural University, Nanchang City (CN)

(72) Inventors: Ziming WU, Nanchang City (CN); Ruicai HAN, Nanchang City (CN); Zhirong XU, Nanchang City (CN); Zujun LI, Nanchang City (CN); Xunfeng HE, Nanchang City (CN); Yongjun ZENG, Nanchang City (CN); Yanhua ZENG, Nanchang City (CN)

(73) Assignee: Jiangxi Agricultural University, Nanchang City (CN)

(21) Appl. No.: 16/948,658

(22) Filed: Sep. 28, 2020

(30) Foreign Application Priority Data
Nov. 22, 2019 (CN) 201911154738.2

(51) Int. Cl. C12N 15/82

(52) U.S. Cl. CPC C12N 15/8261; C12N 15/8205

(57) ABSTRACT

The present invention discloses a gene OsNia3 of a rice nitrate reductase NIA3 protein. The cDNA sequence of the gene is as set out in SEQ ID NO.1, and the rice NIA3 protein encoded by it has an amino acid sequence as set out in SEQ ID NO.2. A homozygous mutant in which the rice gene OsNia3 is knocked out and a homozygous line in which the OsNia3 is over-expressed are obtained by utilizing a transgenic technology. It has been discovered by analysis that the line in which the OsNia3 gene is knocked out has a shortened plant height and a shorted growth period; while the line in which the OsNia3 is over-expressed has a relatively high plant height and a prolonged growth period.

### TECHNICAL FIELD

The present invention relates to the technical field of genetic engineering, and in particular to a gene OsNia3 of a rice nitrate reductase NIA3 protein, and its application.

### BACKGROUND

A plant mainly absorb a nitrogen element from soil in the forms of nitrates and ammonium salts, and nitrate assimilation is a highly regulated process of nitrogen utilization^{[1]}. The first step of nitrate degradation occurs in cytoplasm, in which the nitrate is reduced to a nitrite by a nitrate reductase (NR), the nitrite enters a chloroplast or plastid, and then is utilized by the plant after subjected to a series of subsequent metabolic reactions. At present, it has been found by research that there are two nitrate reductase genes (AtNia1 and AtNia2) in Arabidopsis thaliana, which encode NIA1 and NIA2 proteins, respectively^{[2]}. After AtNia1 and AtNia2 are knocked out, the NR activity of a Arabidopsis thaliana mutant is decreased significantly, while the NR activity could hardly be detected in a nia1nia2 double knockout mutant^{[3]}. Over-expression of the Nia gene in Arabidopsis thaliana can enhance the NR activity of a transgenic line and reduce accumulation of the nitrate in the plant^{[4]}. The NR activity in Arabidopsis thaliana is mainly controlled by the gene AtNia2, and the mutation of the AtNia2 gene will lead to the 90% loss of the NR activity^{[5]}. There are 2 nitrate reductase genes in rice, which are OsNia1 and OsNia2, respectively^{[6]}. In a process of searching OsNia1 mRNA sequence from NCBI (http://www.ncbi.nlm.nih.gov/), it is found that the homology between the mRNA of the OsNia1 gene and the mRNA of [NADH]1-like (LOC4345798) is 99%, and the [NADH]1-like (LOC4345798) is named OsNia3 in subsequent experiments. Studies have shown that knockout and over-expression of the OsNia3 gene can change the plant height and growth period, and affect the growth and development of rice.

The references mentioned above are as follows:

[1] Crawford N M. Nitrate: nutrient and signal for plant growth[J]. Plant Cell, 1995, 7(7): 859-868;

[2] Choi H K, Kleinhofs A, An G Nucleotide sequence of rice nitrate reductase genes [J]. Plant Molecular Biology, 1989(13):731-733;

[3] Wilkinson J Q, Crawford N M. Identification and characterization of a chlorate-resistant mutant of Arabidopsis thaliana with mutations in both nitrate reductase structural genes NIA1 and NIA2[J]. Molecular & general genetics: MGG, 1993, 239(1-2):289;

[4] Curtis I S, Power J B, de Laat A M M, et al. Expression of a chimeric nitrate reductase gene in transgenic lettuce reduces nitrate in leaves[J]. Plant Cell Reports, 1999, 18(11):889-896;

[5] Yu X, Sukumaran S, Márton L. Differential Expression of the Arabidopsis Nia1 and Nia2 Genes1: Cytokinin-Induced Nitrate Reductase Activity Is Correlated With Increased[J]. Plant Physiology, 1998, 116(3):1091-1096;

[6] Hasegawa H, Katagiri T, Ida S, et al. Characterization of a rice (Oryza sativa L.) mutant deficient[J]. Theoretical and Applied Genetics, 1992(84):6-9.

### SUMMARY

The problem solved by the present invention is to provide a gene OsNia3 of a rice nitrate reductase NIA3 protein, and its application, so as to solve the problems in the aforementioned background.

The technical problem solved by the present invention is realized by adopting the following technical solution.

The gene OsNia3 of the rice nitrate reductase NIA3 protein has a cDNA sequence as set out in SEQ ID NO.1.

The amino acid sequence of the rice protein NIA3 encoded by the gene OsNia3 of the rice nitrate reductase NIA3 protein is as set out in SEQ ID NO.2.

The gene OsNia3 of the rice nitrate reductase NIA3 protein is applied to a rice transgenic plant, and the specific steps are as follows.

1) Extraction of Total RNA

The rice variety Kitaake is selected as an extracting material of RNA, rice seedlings are allowed to grow to about 20 d, then the leaves are taken and cryopreserved with liquid nitrogen, thereafter some of the leaves cryopreserved with liquid nitrogen are taken and crushed with a mortar, the total RNA of the rice leaves is extracted according to the instructions of a RNAprep pure Plant Kit, then the concentration and mass of the total RNA are detected by using a NanoDrop microvolume spectrophotometers and fluorometer, and the integrity of the total RNA is detected with 1% agarose gel electrophoresis.

2) Clone of Rice Gene OsNia3

The mRNA sequence of the ONia1 gene is searched from NCBI (http://www.ncbi.nlm.nih.gov/), then it is found that the mRNA of the OsNia1 gene is highly homologous with the mRNA of the [NADH]1-like (LOC4345798) (with the homology of 99%), the [NADH]1-like (LOC4345798) is called OsNia3 in subsequent experiments, and primers P1: SEQ ID NO.3 and P2: SEQ ID NO.4 for two terminals are designed according to the full-length sequence of the rice OsNia3;

SEQ ID NO.3:
5-TGAACGCAGAACCGAACAC-3;
SEQ ID NO. 4:
5-TCCACGGGCCACCATAC-3.

The total RNA of rice leaves obtained in the step 1) is transcribed to synthesize a first chain of the cDNA, PCR amplification is conducted by taking the first chain of the cDNA as a template, extraction is conducted by using a TaKaRa MiniBEST Agarose Gel DNA Extraction Kit, the gene of interest is ligated with an over-expression vector pCUbi1390-GFP to obtain an over-expression vector pCUbi1390-GFP-Nia3, and then sequencing is conducted to obtain the cDNA sequence SEQ ID NO.1 of the rice gene OsNia3 and the amino acid sequence SEQ ID NO.2 of the rice protein NIA3 encoded by the rice gene OsNia3.

3) Construction of Rice Gene OsNia3 Knockout Vector pCas9-OsNia3

A primer fragment is designed according to the full-length cDNA sequence of the rice gene OsNia3 obtained in the step 2) by using a CRISPR-P online website (http://cbi.hzau.edu.cn/cgi-bin/CRISPR), then the sequence of the designed primer fragment and the partial sequence of a sgRNA backbone on a vector pCas9 are together pasted into a RNA fold Web server for RNA structure prediction, such that a suitable primer fragment P3: SEQ ID NO.5 is obtained, then a linker sequence is added onto the primer fragment P3: SEQ ID NO.5 to obtain a primer fragment P4: SEQ ID NO.6 and its reverse complementary sequence P5: SEQ ID NO.7, the primer fragment P4 and the reverse complementary sequence P5 are sent to a company for synthesis, then the obtained primer fragment P4 and its reverse complementary sequence P5 are bound by annealing with a PCR amplifier to obtain an annealed product, finally the annealed product (i.e., the 18 bp sequence of the rice gene OsNia3) is cloned into the vector pCas9, and sequencing is conducted for identification to ensure that the sequence of the gene of interest is correct, thereby obtaining the rice gene OsNia3 knockout vector pCas9-OsNia3;

SEQ ID NO. 5:
5-GCTCGGGGAACCGCCGCA-3;
SEQ ID NO. 6:
5-GCTCGGGGAACCGCCGCAGTTTTAGAGCTATGCTGAAAAGCATAG
CAAGTTAAAATAAGGCTAGTCCGTTATCAACTTGAAAAAGTGGCACCGAG
TCGGTGC-3;
SEQ ID NO. 7:
GCACCGACTCGGTGCCACTTTTTCAAGTTGATAACGGACTAGCCTTA
TTTTAACTTGCTATGCTTTTCAGCATAGCTCTAAAACTGCGGCGGTTCCC
CGAGC-3.

4) Construction of Expression Vector pCUBi1390-GFP-OsNia3 for Rice Gene OsNia3

According to the cDNA sequence SEQ ID NO.1 of the rice gene OsNia3, an upstream primer P6: SEQ ID NO.8 and a downstream primer P7: SEQ ID NO.9 which carry restriction enzyme cleavage sites Hand III and BamH I that are completely encoded at the amplification position, are designed;

SEQ ID NO. 8:
5-TCTGCACTAGGTACCTGCAGATGGCTGCTTCCGTGC-3;
SEQ ID NO. 9:
5-ATGGATCCGTCGACCTGCAGGAACACGATGAAAGAATTGGCC-3;

after PCR amplification is conducted by taking the over-expression vector pCUbi1390-GFP-Nia3 obtained in the step 2) as a template, the cDNA of the rice gene OsNia3 is cloned to the enzyme cleavage sites Hand III and BamH I in the binary expression vector pCUBi1390-GFP, and sequencing is conducted for identification to ensure that the reading frame of a coding region in the expression vector is correct, thereby obtaining the rice gene OsNia3 over-expression vector pCUBi1390-GFP-OsNia3.

5) Genetic Transformation of Rice

The rice gene OsNia3 knockout vector pCas9-OsNia3 obtained in the step 3) is transferred into Agrobacterium tumefaciens and further transferred into the japonica rice variety Kitaake, and the transgenic plant is identified by PCR and sequencing to obtain a homozygous knockout mutant nia3 having mutation of the OsNia3 gene and no backbone of the vector, wherein the rice mutant nia3 shows a reduced plant height and a shortened growth period;

the expression vector pCUBi1390-GFP-OsNia3 of the rice gene OsNia3, as obtained in the step 4), is transferred into Agrobacterium tumefaciens and further transferred into the homozygous knockout mutant nia3, and the transgenic plant is verified by PCR and RT-PCR to obtain a homozygous OsNia3 over-expression line which shows an increased plant height and a prolonged growth period.

Beneficial Effects:

1) the present invention discloses the gene OsNia3 of the rice nitrate reductase NIA3 protein and the protein encoded thereby, the gene OsNia3 of the rice nitrate reductase NIA3 protein is reported for the first time in rice, and transgenic experiments prove that knockout and over-expression of this gene can affect plant height and growth period of rice, so this gene is expected to be introduced into a plant as a gene of interest to improve the nitrogen utilization efficiency of the plant and conduct improvement of rice;

2) in the present invention, the obtained transgenic line is subjected to PCR molecular identification and sequencing verification to obtain the homozygous knockout line and over-expression line of the rice gene OsNia3, then the growth and development of the transgenic rice are analyzed, and it is found that the OsNia3 gene knockout line has a reduced plant height and a shortened growth period, while the OsNia3 over-expression line has a higher plant height and a prolonged growth period.

### DESCRIPTION OF THE EMBODIMENTS

In order to make the technical means, creative features, goals and effects achieved by the present invention easy to understand, the present invention will be further explained with specific embodiments.

The gene OsNia3 of the rice nitrate reductase NIA3 protein is applied to a rice transgenic plant, and the specific steps are as follows.

1) Extraction of Total RNA

The rice variety Kitaake is selected as an extracting material of RNA, rice seedlings are allowed to grow to about 20 d, then the leaves are taken and cryopreserved with liquid nitrogen, thereafter some of the leaves cryopreserved with liquid nitrogen are taken and crushed with a mortar, the total RNA of the rice leaves is extracted according to the instructions of a RNAprep pure Plant Kit, then the concentration and mass of the total RNA are detected by using a NanoDrop microvolume spectrophotometers and fluorometer, and the integrity of the total RNA is detected with 1% agarose gel electrophoresis.

2) Clone of Rice Gene OsNia3

The mRNA sequence of the Nia1 gene is searched from NCBI (http://www.ncbi.nlm.nih.gov/), then it is found that the mRNA of the Nia1 gene is highly homologous with the mRNA of the [NADH]1-like (LOC4345798) (with the homology of 99%), the [NADH]1-like (LOC4345798) is named OsNia3 in subsequent experiments, and primers P1: SEQ ID NO.3 and P2: SEQ ID NO.4 for two terminals are designed according to the full-length sequence of the rice OsNia3;

SEQ ID NO. 3:
5-TGAACGCAGAACCGAACAC-3;
SEQ ID NO. 4:
5-TCCACGGGCCACCATAC-3;

the total RNA of rice leaves obtained in the step 1) is transcribed to synthesize a first chain of the cDNA, PCR amplification is conducted by taking the first chain of the cDNA as a template, extraction is conducted by using a TaKaRa MiniBEST Agarose Gel DNA Extraction Kit, the gene of interest is ligated with an over-expression vector pCUbi1390-GFP to obtain an over-expression vector pCUbi1390-GFP-Nia3, and then sequencing is conducted to obtain the cDNA sequence SEQ ID NO.1 of the rice gene OsNia3 and the amino acid sequence SEQ ID NO.2 of the rice protein NIA3 encoded by the rice gene OsNia3.

3) Construction of Rice Gene OsNia3 Knockout Vector pCas9-OsNia3

A primer fragment is designed according to the full-length cDNA sequence of the rice gene OsNia3 obtained in the step 2) by using a CRISPR-P online website (http://cbi.hzau.edu.cn/cgi-bin/CRISPR), then the sequence of the designed primer fragment and the partial sequence of a sgRNA backbone on a vector pCas9 are together pasted into a RNA fold Web server for RNA structure prediction, such that a suitable primer fragment P3: SEQ ID NO.5 is obtained, then a linker sequence is added onto the primer fragment P3: SEQ ID NO.5 to obtain a primer fragment P4: SEQ ID NO.6 and its reverse complementary sequence P5: SEQ ID NO.7, the primer fragment P4 and its reverse complementary sequence P5 are sent to a company for synthesis, then the obtained primer fragment P4 and the reverse complementary sequence P5 are bound by annealing with a PCR amplifier to obtain an annealed product, finally the annealed product (i.e., the 18 bp sequence of the rice gene OsNia3) is cloned into the vector pCas9, and sequencing is conducted for identification to ensure that the sequence of the gene of interest is correct, thereby obtaining the rice gene OsNia3 knockout vector pCas9-OsNia3;

SEQ ID NO. 5:
5-GCTCGGGGAACCGCCGCA-3;
SEQ ID NO. 6:
5-GCTCGGGGAACCGCCGCAGTTTTAGAGCTATGCTGAAAAGCATAG
CAAGTTAAAATAAGGCTAGTCCGTTATCAACTTGAAAAAGTGGCACCGAG
TCGGTGC-3;
SEQ ID NO. 7:
5-GCACCGACTCGGTGCCACTTTTTCAAGTTGATAACGGACTAGCCTT
ATTTTAACTTGCTATGCTTTTCAGCATAGCTCTAAAACTGCGGCGGTTCC
CCCGAGC-3.

4) Construction of Expression Vector pCUBi1390-GFP-OsNia3 for Rice Gene OsNia3

According to the cDNA sequence SEQ ID NO.1 of the rice gene OsNia3, an upstream primer P6: SEQ ID NO.8 and a downstream primer P7: SEQ ID NO.9 which carry restriction enzyme cleavage sites Hand III and BamH I that are completely encoded at the amplification position, are designed;

SEQ ID NO. 8:
5-TCTGCACTAGGTACCTGCAGATGGCTGCTTCCGTGC-3;
SEQ ID NO. 9:
5-ATGGATCCGTCGACCTGCAGGAACACGATGAAAGAATTGGCC-3;

after PCR amplification is conducted by taking the over-expression vector pCUbi1390-GFP-Nia3 obtained in the step 2) as a template, the cDNA of the rice gene OsNia3 is cloned to the enzyme cleavage sites Hand III and BamH I in the binary expression vector pCUBi1390-GFP, and sequencing is conducted for identification to ensure that the reading frame of a coding region in the expression vector is correct, thereby obtaining the rice gene OsNia3 expression vector pCUBi1390-GFP-OsNia3.

5) Genetic Transformation of Rice

The rice gene OsNia3 knockout vector pCas9-OsNia3 obtained in the step 3) is transferred into Agrobacterium tumefaciens and further transferred into the japonica rice variety Kitaake, and the transgenic plant is identified by PCR and sequencing to obtain a homozygous knockout mutant nia3 having mutation of the OsNia3 gene and no backbone of the vector, wherein the rice mutant nia3 shows a reduced plant height and a shortened growth period;

the expression vector pCUBi1390-GFP-OsNia3 of the rice gene OsNia3, as obtained in the step 4), is transferred into Agrobacterium tumefaciens and further transferred into the homozygous knockout mutant nia3, and the transgenic plant is verified by PCR and RT-PCR to obtain a homozygous OsNia3 over-expression line which shows an increased plant height and a prolonged growth period.

1-10. (canceled)

11. A rice gene OsNia3 knockout vector pCas9-OsNia3 obtained by connecting an 18 bp sequence of the gene OsNia3 of a rice nitrate reductase NIA3 protein to a vector pCas9.

12. A rice gene OsNia3 expression vector pCUBi1390-GFP-OsNia3 obtained by cloning a cDNA sequence of a rice gene OsNia3 as set out in SEQ ID NO. 1 into enzyme cleavage sites Hand III and BamH I in a binary expression vector pCUBi1390-GFP.

13. A method of producing a transgenic rice plant, comprising:
transferring a rice gene OsNia3 knockout vector pCas9-OsNia3 into Agrobacterium tumefaciens; and
transferring the Agrobacterium tumefaciens into a japonica rice variety Kitaake to obtain a homozygous knockout mutant nia3 having mutation of the OsNia3 gene and no backbone of the knockout vector.

14. The method of producing a transgenic rice plant of claim 13, further comprising:
producing the rice gene OsNia3 knockout vector, by:
designing a primer fragment according to a full-length cDNA sequence of the rice gene OsNia3 as set out in SEQ ID NO. 1 using a CRISPR-P online website, P1 having SEQ ID NO.3 and P2 having SEQ ID NO.4 for two terminals;
pasting the sequence of the designed primer fragment and a partial sequence of a sgRNA backbone on a vector pCas9 into an RNA fold Web server for RNA structure prediction, such that a primer fragment P3: SEQ ID NO.5 is obtained;
adding a linker sequence onto the primer fragment P3: SEQ ID NO.5 to obtain a primer fragment P4: SEQ ID NO.6 and its reverse complementary sequence P5: SEQ ID NO.7:
synthesizing the primer fragment P4 and its reverse complementary sequence P5;
annealing the obtained primer fragment P4 and its reverse complementary sequence P5 with a PCR amplifier to obtain an annealed product; and
cloning the annealed product into the vector pCas9.

15. The method of producing a transgenic rice plant of claim 14, wherein the primers for two terminals have the sequences
SEQ ID NO. 3:
5-TGAACGCAGAACCGAACAC-3;
and
SEO ID NO. 4:
5-TCCACGGGCCACCATAC-3.

16. The method of producing a transgenic rice plant of claim 14, wherein the primer fragments have the sequences
SEQ ID NO. 5:
5-GCTCGGGGAACCGCCGCA-3;
SEQ ID NO. 6:
5-GCTCGGGGAACCGCCGCAGTTTTAGAGCTATGCTGAAAAGCATAG
CAAGTTAAAATAAGGCTAGTCCGTTATCAACTTGAAAAAGTGGCACCG
AGTCGGTGC-3;
and
SEQ ID NO. 7:
5-GCACCGACTCGGTGCCACTTTTTCAAGTTGATAACGGACTAGCCTT
ATTTTAACTTGCTATGCTTTTCAGCATAGCTCTAAAACTGCGGCGGTTC
CCCGAGC-3.

17. The method of producing a transgenic rice plant of claim 13, further comprising:
transferring an expression vector for a rice gene OsNia3 pCUBi1390-GFP-OsNia3 into Agrobacterium tumefaciens; and
transferring the Agrobacterium tumefaciens into the homozygous knockout mutant nia3.

18. The method of producing a transgenic rice plant of claim 17, further comprising:
producing the expression vector for the rice gene OsNia3 pCUBi1390-GFP-OsNia3, by:
designing an upstream primer P6: SEQ ID NO.8 and a downstream primer P7: SEQ ID NO.9 which carry restriction enzyme cleavage sites Hand III and BamH I that are completely encoded at the amplification position;
amplifying an over-expression vector pCUbi1390-GFP-Nia3 by PCR; and
cloning cDNA of the rice gene OsNia3 to the enzyme cleavage sites Hand III and BamH I in a binary expression vector pCUBi1390-GFP.

19. The method of producing a transgenic rice plant of claim 18, further comprising:
producing the over-expression vector pCUbi1390-GFP-Nia3, by:
extracting total RNA of rice leaves;
designing primers P1 having SEQ ID NO.3 and P2 having SEQ ID NO.4 for two terminals according to a full-length sequence of the rice gene OsNia3;
transcribing the total RNA of rice leaves to synthesize a first chain of cDNA;
amplifying the first chain of the cDNA by PCR;
extracting the cDNA using a TaKaRa MiniBEST Agarose Gel DNA Extraction Kit; and
ligating the cDNA with an over-expression vector pCUbi1390-GFP.

20. The method of producing a transgenic rice plant of claim 19, wherein the primers for two terminals have the sequences
SEQ ID NO. 3:
5-TGAACGCAGAACCGAACAC-3;
and
SEQ ID NO. 4:
5-TCCACGGGCCACCATAC-3.

21. The method of producing a transgenic rice plant of claim 18, wherein the upstream and downstream primers have the sequences
SEQ ID NO. 8:
5-TCTGCACTAGGTACCTGCAGATGGCTGCTTCCGTGC-3;
and
SEQ ID NO. 9:
5-ATGGATCCGTCGACCTGCAGGAACACGATGAAAGAATTGGCC-3.


SEQUENCE LISTING (9 sequences)

SEQ ID NO: 1 - DNA, 3315 bp, artificial sequence
  cDNA sequence of rice gene OsNia3
gcgcctcttccaggccactacttctctgggctctggctactcttcgacggcggccggcgtagcgagctgtagctgagctgaacgcagaaccgaacaccaagcatggccgcttccgtgcagccgcggcagttcggccacctcgagccgggctccgcgccggtgtgcggcgccgcatcctcgaacggcgccaaggcgtaccctcccgcgaacggcatcccgcgccgcgccgactcaccggtgcgcgggtgcggcttccctcccctcgtctcgccaccttcgcggaagccgcccagcgatgggtcggacgacgaggaggaggagcaggaggactggcgggagctgtacggctcgcacctgcagctggaggtggaaccgtcggtgcgcgacgcgcgcgacgagggcaccgccgacgcgtggatcgagcgcaacccgttgctgatccggctcaccgggaaacacccgctgaactgcgaggcgccgctggcgaggctcatgcaccacggcttcatcaccccggctgcgctgcacttcgtgcgcaaccacggcgcagtgccgcggggtgactggtcgacgtggaccgtcgaggtgacggggctcgtcaagcgtcccatgcggctcaccgtggacgagctggtcaacggcttccccgccgtggaggtccccgtcacgctggcctgctcggggaaccgccgcaaggagcagaacatggtgcagcagactgtggggttcaactttggcgccgccgccgtgtccacgtcggtgtggcacggcgcccgcctccgcgacgtgctccggcggtgcggcatcatgcccagcaagggcggtgcgctcaacgtgtgcttcgagggcgccgaggacctccccggcggcggcggctccaagtacggcaccagcatcacacgccagtgggcgctggacccgtcgcgggacatcatgctcgcctacatgcagaatggcgagccgctgctccccgaccacggcttccccgtccgcgccatcatccccggctgcaccggcggccgcatggtcaagtgggtcaagcgcatcatcgtcaccaccgccgagtccgacaactactaccattacaaggacaaccgcgtcttcccgtcccatgtcgacgccgagctcgccaacgccgatgcgtggtggtacaagccggagtacatcatcaacgagctgaacgtgaactcggtgatcacggcgcccgggcacgacgagatcctgcccatcaacggcatcaccacgcagcgcggctacaccatgaagggatacgcctactccggcggcggcaagaggatcacgcgggtggaggtgacgctggacggcggcgagacatggctggtgtgcgtgctggacctcccggagaagcccaccaagtacggcaagcactggtgctggtgcttctggtccgtcgaggtcgaggtgctcgacctcctcggcgccaaggagatcgccgtgcgcgcctgggaccagtcgcacaacacccagcccgagaagctcatctggaatctcatggggatgatgaacaactgctggttcaaggtgaaggtgaacgtgtgccggccgcacaagggtgagatcgggctggtgttcgagcacccgacgcagcccgggaaccagaccggcgggtggatggcgaggcagaagcacctggagacggcggaggcggccgcaccggggctgaagcggagcacgtcgacgccgttcatgaacaccaccgacggcaagcagtttaccatgtccgaggtgcgcaagcactcgtcgcaggactcggcgtggatcgtcgtccacggtcacgtctacgactgcacggccttcctcaaggaccaccccggcggcgccgacagcatcctcatcaacgccggcaccgactgcaccgaggagttcgacgccatccactccgacaaggccaaggcgctcctcgacacctaccgcatcggcgagctcatcaccaccggcgccgggtacagctccgacaactccgtccacggcgcgtccaacctctcccagctcgcccccatccgcgaggccatcaaggcgccggcgcccgtcgcgctctccagcccgcgcgacaaggtcccctgccaactcgtcgacaagaaggagctctcccgcgacgtccgcctcttccgcttcgcgctgccgtcctccgaccaggtgctcggcctccccgtcggcaagcacatcttcgtgtgcgccagcatcgaagggaagctgtgcatgcgggcgtacacgccgacgagcatggtcgacgaggtcggccacttcgacctcctcatcaaggtgtacttcaagaacgagcaccccaagttccccgatggcgggctcatgacgcagtacctggactcgctccccgtgggcgcctacatcgacgtcaaggggccactcggccacgtcgagtacaccggccgcggcgagttcgtcatcaacggcaagccgcggaacgcgcggcggctggcgatgatcgccggcgggagcgggatcacgcccatgtaccaggtcatccagtcggtgctgcgcgaccagccggaggacacgacggagatgcacctggtgtacgcgaaccggacggaggacgacatcctcctccgcgacgagctcgaccggtgggcggcggagtacccggacaggctcaaggtgtggtacgtcatcgaccaggtgaagcggccggaggaagggtggaagtacggcgtcgggttcgtcacggaggaggtgctgcgggagcacgtgccggagggcggcgacgacacgctcgcgctcgcctgcgggccgccgccgatgatcaagttcgccgtctcgccgaacctggagaagatgaagtacgacatggccaattctttcatcgtgttctaaactcctactactaaattatacgcaccgcattaattattttggtatacgtacgacgatgtatatacgtgtcgtgccaattagaaacgaaatgctgcatgcatgcgtatgcaagatgagagaaaaaaaaacaatatctaagttgtagtagtgtactcttgataatatcctagagtacatactgcatacataggccatgatttccctttctaagaggctagcggttgattccttcctgtatactatgttacgaaccaaggggggattgggaattttggattgaacgagcagaggaagaatagaaacagaggaagaactcggaagaacagagtagtttgggagagaagcagtaggattattcagagatagtgttttgattacaaactagtatggtggcccgtggagattgcgcggctagcatcattatattttctctcatataattacatatatgatttctcatta

SEQ ID NO: 2 - protein, 916 aa, artificial sequence
  The amino acid sequence of the rice protein NIA3 encoded by the gene OsNia3 of the rice nitrate reductase NIA3 protein
MAASVQPRQFGHLEPGSAPVCGAASSNGAKAYPPANGIPRRADSPVRGCGFPPLVSPPSRKPPSDGSDDEEEEQEDWRELYGSHLQLEVEPSVRDARDEGTADAWIERNPLLIRLTGKHPLNCEAPLARLMHHGFITPAALHFVRNHGAVPRGDWSTWTVEVTGLVKRPMRLTVDELVNGFPAVEVPVTLACSGNRRKEQNMVQQTVGFNFGAAAVSTSVWHGARLRDVLRRCGIMPSKGGALNVCFEGAEDLPGGGGSKYGTSITRQWALDPSRDIMLAYMQNGEPLLPDHGFPVRAIIPGCTGGRMVKWVKRIIVTTAESDNYYHYKDNRVFPSHVDAELANADAWWYKPEYIINELNVNSVITAPGHDEILPINGITTQRGYTMKGYAYSGGGKRITRVEVTLDGGETWLVCVLDLPEKPTKYGKHWCWCFWSVEVEVLDLLGAKEIAVRAWDQSHNTQPEKLIWNLMGMMNNCWFKVKVNVCRPHKGEIGLVFEHPTQPGNQTGGWMARQKHLETAEAAAPGLKRSTSTPFMNTTDGKQFTMSEVRKHSSQDSAWIVVHGHVYDCTAFLKDHPGGADSILINAGTDCTEEFDAIHSDKAKALLDTYRIGELITTGAGYSSDNSVHGASNLSQLAPIREAIKAPAPVALSSPRDKVPCQLVDKKELSRDVRLFRFALPSSDQVLGLPVGKHIFVCASIEGKLCMRAYTPTSMVDEVGHFDLLIKVYFKNEHPKFPDGGLMTQYLDSLPVGAYIDVKGPLGHVEYTGRGEFVINGKPRNARRLAMIAGGSGITPMYQVIQSVLRDQPEDTTEMHLVYANRTEDDILLRDELDRWAAEYPDRLKVWYVIDQVKRPEEGWKYGVGFVTEEVLREHVPEGGDDTLALACGPPPMIKFAVSPNLEKMKYDMANSFIVF

SEQ ID NO: 3 - DNA, 19 bp, artificial sequence
  P1
tgaacgcagaaccgaacac

SEQ ID NO: 4 - DNA, 17 bp, artificial sequence
  P2
tccacgggccaccatac

SEQ ID NO: 5 - DNA, 18 bp, artificial sequence
  P3
gctcggggaaccgccgca

SEQ ID NO: 6 - DNA, 102 bp, artificial sequence
  P4
gctcggggaaccgccgcagttttagagctatgctgaaaagcatagcaagttaaaataaggctagtccgttatcaacttgaaaaagtggcaccgagtcggtgc

SEQ ID NO: 7 - DNA, 102 bp, artificial sequence
  P5
gcaccgactcggtgccactttttcaagttgataacggactagccttattttaacttgctatgcttttcagcatagctctaaaactgcggcggttccccgagc

SEQ ID NO: 8 - DNA, 36 bp, artificial sequence
  P6
tctgcactaggtacctgcagatggctgcttccgtgc

SEQ ID NO: 9 - DNA, 42 bp, artificial sequence
  P7
atggatccgtcgacctgcaggaacacgatgaaagaattggcc